(19) United States
(12) Patent Application Publication
(10) Pub. No.: US 2015/0230958 A1
(43) Pub. Date: Aug. 20, 2015

(54) INTERNAL COVERING MEMBRANE OF DUODENUM PREPARED BY ELECTROSPINNING METHOD

(71) Applicant: Ping Wan, Shanghai (CN)

(72) Inventor: Ping Wan, Shanghai (CN)

(21) Appl. No.: 14/422,890

(22) PCT Filed: Aug. 18, 2013
(86) PCT No.: PCT/CN2013/081711

§ 371 Date: Feb. 20, 2015

(30) Foreign Application Priority Data
Aug. 21, 2012 (CN) 201210298363.9

(51) Int. Cl. A61F 5/00

(52) U.S. Cl. CPC A61F 5/0076

(57) ABSTRACT

An internal covering membrane of duodenum can be made from biocompatible materials via electrospinning technology is described herein. After the internal covering membrane of duodenum is implanted into the duodenum, it can not only prevent food from contacting with the intestinal mucosa in anatomy, but also make no affect on the functions of intestinal mucosa cells in physiology. The internal covering membrane can be made by blending electrospinning or/and multilayer electrospinning or/and core-shell electrospinning or/and dry electrospinning, and can be used to manufacture medical devices for treating diabetes and obesity, with functions of reducing damage, preventing falling-off, avoiding removal and inhibiting bounce.

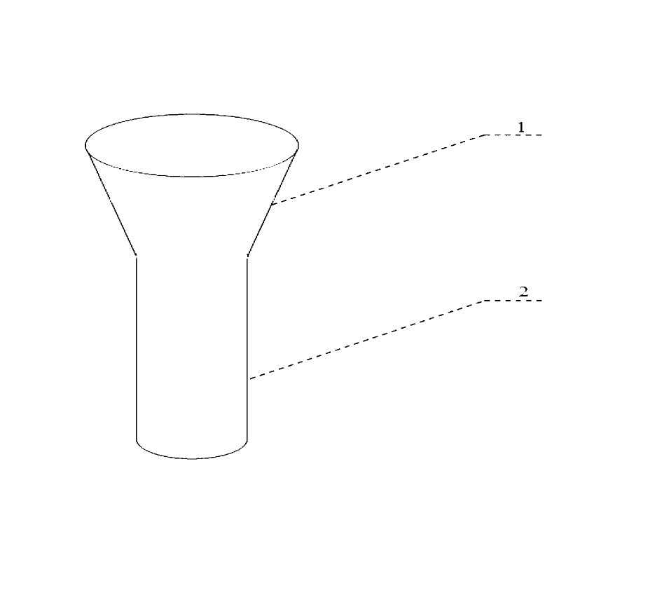

### FIELD OF THE INVENTION

The present invention relates to a degradable medical device internally built in a digestive tract and, more particularly, to an internal covering membrane of duodenum for treating diabetes and obesity.

### BACKGROUND OF THE INVENTION

In March of 2011, at the 2nd International Type 2 Diabetes Intervention Treatment Conference held in New York, USA, the International Diabetes Federation (IDF) issued a statement for the first time, declaring that the gastric bypass operation can be used for treating obese patients with type 2 diabetes and can reduce the occurrence and development of chronic complications of diabetes mellitus (Chinese Medical Sciences, 2011, 1(22):1-2). This operation can also obviously improve the complications such as hypertension, obesity and dyslipidemia of patients (Chinese Medical Sciences, 2011, 1(21):3-5).

But the gastric bypass operation has clinical risks such as death, intestinal obstruction, anastomotic leakage, pulmonary embolism, deep venous thrombosis, portal vein injury, respiratory system, etc. (Chinese Journal of Diabetes Mellitus, 2011, 3(3):205-208).

Therefore, in the treatment of diabetes and obesity, the implantation of internal covering membrane of duodenum into a body tends to replace the above-mentioned “gastric bypass operation”.

However, when considering that the internal covering membrane of duodenum obviously prevents food from contacting with the intestinal mucosa in anatomy, the hidden problem that whether it hinders the function of intestinal mucosa cells or not in physiology should not be ignored.

In the invention patent of the prior art “A duodenal casing and a conveyor thereof” (application date: Apr. 9, 2010, date of authorized announcement: Jan. 11, 2012), “the material of the outer casing is an elastic membrane” and “such as a latex membrane”, which do not describe how to prepare the membrane. In the utility model patent of the prior art “A duodenum jejunumbuilt-in casing” (application date: Dec. 6, 2010, date of authorized announcement: Sep. 28, 2011), “the flexible tube consists of a smooth, soft and dense membrane”, “the membrane is a soft membrane” and “the preferred material is fluorine plastic”, which do not describe how to prepare the membrane, either. Further, the two prior patents both do not mention whether the materials used in manufacturing are biocompatible and degradable or not. In the invention patent of the prior art “An internal covering membrane of duodenum made by degradable and biocompatible materials and applications thereof” (application date: May 5, 2012, publication date: Aug. 8, 2012), although its materials are degradable and biocompatible, the invention also does not describe how to manufacture the membrane by electrospinning

In the invention patent of the prior art “Dry electrospinning forming methods of high polymer/ionic liquid spinning solution system” (application date: Apr. 21, 2009, date of authorized announcement: Aug. 31, 2011), it does not mention the application, let alone its application in medical devices.

In the invention patent of the prior art “A method for preparing breathable and waterproof polyurethane nanofiber membrane” (application date: Dec. 5, 2011, publication date: Jun. 27, 2012), it is an electrospinning method of multi nozzles. Although it mentions that it can be applied to “biological tissue engineering”, it has no related embodiments, let alone a specific embodiment about preparing the internal covering membrane of duodenum by the electrospinning method.

In the invention patent of the prior art “A medical covered stent and a preparing method thereof” (application date: Nov. 19, 2011, publication date: May 2, 2012), it uses the electrospinning technology to prepare the inner and outer membranes of a medical stent. Further, its motivation, effect, reason and result are all completely different from those of the present invention.

### SUMMARY OF THE INVENTION

### Technical Problems to be Solved by the Present Invention

In the invention patent of the prior art “A duodenal casing and a conveyor thereof” (application date: Apr. 9, 2010, date of authorized announcement: Jan. 11, 2012), “the material of the outer casing is an elastic membrane” and “such as a latex membrane”, which do not describe how to prepare the membrane. In the utility model patent of the prior art “A Duodenum jejunum built-in casing” (application date: Dec. 6, 2010, date of authorized announcement: Sep. 28, 2011), “the flexible tube consists of a smooth, soft and dense membrane”, “the membrane is a soft membrane” and “the preferred material is fluorine plastic”, which do not describe how to prepare the membrane, either. Further, the two prior patents both do not mention whether the materials used in manufacturing are biocompatible and degradable or not. All parts of the internal covering membrane of duodenum in the present invention are made of biocompatible materials, namely, the present invention solves the biocompatibility problem of the materials implanted into the body and weakens the host response caused by the implantation. After being implanted into the body, the materials can be gradually degraded in the body after 2 months to 5 years, and can be used to manufacture medical devices for treating diabetes and obesity, with functions of reducing damage, preventing falling-off, avoiding removal and inhibiting bounces. In the invention patent of the prior art “An internal covering membrane of duodenum made by degradable and biocompatible materials and applications thereof” (application date: May 5, 2012, publication date: Aug. 8, 2012), although its materials are degradable and biocompatible, it does not describe how to prepare the membrane by electrospinning In the invention patent of the prior art “Dry electrospinning forming methods of high polymer/ionic liquid spinning solution system” (application date: Apr. 21, 2009, date of authorized announcement: Aug. 31, 2011), it does not mention the application, let alone its application in medical devices. In the invention patent of the prior art “A method for preparing breathable and waterproof polyurethane nanofiber membrane” (application date: Dec. 5, 2011, publication date: Jun. 27, 2012), it is an electrospinning method of multi nozzles. Although it mentions that it can be applied to “biological tissue engineering”, it has no related embodiments, let alone a specific embodiment about preparing the internal covering membrane of duodenum by the electrospinning method. In the invention patent of the prior art “A medical covered stent and a preparing method thereof” (application date: Nov. 19, 2011, publication date: May 2, 2012), it uses the electrospinning technology to prepare the inner and outer membranes of a medical stent. Further, its motivation, effect, reason and result are all completely different from those of the present invention.

As mentioned above, the gastric bypass operation can be used for treating obese patients with type 2 diabetes and obviously improve complications such as hypertension, obesity and dyslipidemia of the patients. In the treatment of diabetes and obesity, it's a trend to implant internal covering membrane of duodenum into a body to replace the above-mentioned “gastric bypass operation”. However, when considering that the internal covering membrane of duodenum obviously prevents food from contacting with the intestinal mucosa in anatomy, the hidden problem that whether it hinders the function of intestinal mucosa cells or not in physiology should not be ignored. The intestinal mucosa epithelial cells consist of absorbing cells, goblet cells and Paneth cells, etc., wherein the cells are connected in ways of tight connection, gap connection, adhesion connection and desmosome connection, etc. The intestinal mucosa epithelial cells and the connection between cells make up an intestinal micro-ecological environment and a significant barrier for maintaining a stable environment in the body (including mechanical, chemical, biological and immune barriers), e.g., the side faces of absorbing cells and plasma membrane connect with adjacent cells near the enteric cavity to form a complex of tight connection, which only allows water molecules and small-molecule water-soluble substances to selectively pass through; the goblet cells secrete mucous glycoprotein, which can prevent the digestive enzymes in the digestive tract and hazardous substance from damaging the epithelial cells; Paneth cells have a certain ability of swallowing bacteria, and can secrete lysozyme, natural antibiotic peptide, human defensins 5 and human defensins 6, etc. If only considering of the prevention of food from connecting with the intestinal mucosa obviously seen in anatomy and completely covering the intestinal mucosal surface tightly, due to many deep-seated factors such as oxygen deficit, acid poisoning, oxygen radical, inflammatory medium and so on, the intestinal mucosa may result in cell damage, necrosis, mechanical barrier damage and permeability increase. Further due to the accompanying intestinal dysbacteriosis, bacteria and endotoxin translocation, inflammatory reaction of the intestinal mucosa and immunologic tissue in the mesentery, the intestinal mucosa may be further damaged, the permeability of the intestine may be increased, and bacterial translocation may be promoted, thereby forming a vicious circle and finally resulting in SIRS (Systemic Inflammatory Response Syndrome) and even MODS (Multiple Organ Dysfunction Syndrome). The internal covering membrane of duodenum in the present invention is prepared by electrospinning technology, which can not only prevent food from connecting with the intestinal mucosa in anatomy, but also make no effect on the function of intestinal mucosa cells as far as possible in physiology.

### Technical Solutions of the Present Invention

The invention provides an internal covering membrane of duodenum, and the membrane can be prepared by biocompatible materials via electrospinning, being able to prevent food from contacting with the intestinal mucosa in anatomy without affecting the physiological functions of intestinal mucosa cells.

The internal covering membrane of duodenum may include an ampulla portion and a tube portion, and its thickness may be 1 μm-1 mm.

The diameter and length of the tube portion match with the duodenum and jejunum of different crowds. The diameter is 10-60 mm, and the length matches with that of the duodenum, and can be extended to the jejunum which follows the duodenum, with a length of 80-700 mm.

The ampulla portion is a trumpet-shaped part that follows the tube portion. The ampulla portion can also be columnar, spherical or waist-drum shaped, with the height of 6 mm-100 mm, and the trumpet-shaped part which follows the tube portion is in a gradually opening acute angle, which is 5° C.-45° C. Its thickness, height and angle match with different crowds. An outer surface circumference of the ampulla portion is provided with elastic fibers with anchoring means.

The internal covering membrane of duodenum can be produced by the following: devices and instruments being: a high-voltage electrostatic generator, a micro-injection pump, 1-10 spinning nozzles, a roller or a plate receiving device, a fiber fineness meter, a rotary viscometer, a surface tension meter, a conductivity meter, a digital vacuum scanning electron microscope, and a fume cupboard.

Solution preparation being: preparing medical polylactic acid solution with a concentration of 1 wt %-35 wt %, wherein a solvent is a mixture of chloroform and ethanol, with a volume ratio of 25/75(v/v)-100/0(v/v); preparing medical polyurethane solution with a concentration of 1 wt %-45 wt %, wherein the solvent is dimethyl sulfoxide.

Technological parameter being: spinning voltage: 10-36 kV, advance speed: 0.1-3.5 ml/h, acceptable distance: 10-28 cm, inner diameter of pinhole: 0.1-1 mm.

Electrospinning process being: flattening a needle head, in the fume cupboard fixing a syringe injected with electrospinning solution on the micro-injection pump, connecting the output cable of the high-voltage electrostatic generator to the metal needle on the front end of the syringe, making the receiving device grounded, adjusting the distance between the receiving device and a capillary, starting the micro-injection pump, adjusting a flow rate of the injection pump, gradually increasing the voltage after a stable hemispherical droplet, that is Taylor cone, is formed in the capillary orifice, and collecting electrospinning fibers in the form of a non-woven membrane on the receiving device. The tube portion is able to use medical polyurethane solution for electrospinning and the ampulla portion is able to use medical polylactic acid solution for electrospinning, and the thickness of the internal covering membrane can be 1 μm-1 mm.

Solution determination being: performing water bathing at constant temperature of 25° C., rotating the viscometer, choosing a proper rotor, and measuring the viscosity of the electro-spinning solution with a unit of centipoises (cP); measuring the surface tension of the solution at room temperature with the surface tension meter with a unit of mN/m; and measuring the conductivity of the solution at the room temperature with the conductivity meter, wherein the conductivity electrodes are platinum electrodes, the electrode constant is 0.99, and the unit is mS/cm.

Representation of electrospinning fibers being: cutting samples to pieces of 5×5 cm, plating gold on the surface, observing with the scanning electron microscope, measuring the diameter of the electrospinning fibers on the scanning electron microscope photos with Adobe Photoshop 9.0, measuring all the fibers or different parts of one fiber on the photos, calculating an average fiber diameter and diameter distribution; and measuring the crystal property, surface contact angle, and mechanical property with a material testing machine.

The air permeability of the internal covering membrane material of duodenum can be 0.3-0.9 cm³/cm²/s, and its moisture permeability can be 28-42 cmH₂O.

The electrospinning solution of the internal covering membrane of duodenum can be prepared by the following high polymer materials: polylactic acid, polyurethane, PEUR Poly(ether urethane), polyether sulfone, polyglutamic acid, polyvinyl alcohol, polyhydroxybutyrate, caprolactone, polycaprolactone, polyhydroxybutyrate, polyvinylpyrrolidone, poly-L-lactide, recombinant spider silk protein, amino acids, polycaprolactone, caprolactam, hydroxyapatite, elastin, heparin, and glycolic acid, and blending modification is performed to satisfy degradation speed, degradation cycle, elasticity of the membrane, strength of the membrane, specific surface area, porosity and other specific requirements.

The internal covering membrane of duodenum can be prepared by blending electrospinning or/and multilayer electrospinning or/and core-shell electrospinning or/and dry electrospinning.

The internal covering membrane of duodenum can not only prevent food from contacting with the intestinal mucosa in anatomy, but also make no affect on the function of intestinal mucosa cells in physiology.

The internal covering membrane of duodenum can be used to manufacture medical devices for treating diabetes and obesity, with functions of reducing damage, preventing falling-off, avoiding removal and inhibiting bounce.

### Beneficial Effects of the Present Invention

The invention provides an internal covering membrane of duodenum prepared from degradable and biocompatible materials by electrospinning technology, which is implanted into the duodenum. Comparing with the prior art (the invention patent “A duodenal casing and a conveyor thereof”, the utility model patent “A duodenum jejunumbuilt-in casing”, and the invention patent “An internal covering membrane of duodenum made by degradable and biocompatible materials and applications thereof”), the internal covering membrane of duodenum in the present invention can not only prevent food from contacting with the intestinal mucosa in anatomy, but also make no affect on the functions of intestinal mucosa cells in physiology, and the medical devices for treating diabetes and obesity made by the membrane can reduce damage, prevent falling-off, avoid removal and inhibit bounce.

### BRIEF DESCRIPTION OF THE DRAWING

The figure is a schematic diagram according to embodiments of the invention.

The parts or portions marked in the figure are an ampulla portion 1 and a tube portion 2.

### DETAILED DESCRIPTION OF THE EMBODIMENTS

Further illustration of the present invention will be made in details in connection with specific examples.

### Embodiment One

Medical polylactic acid solution is prepared by a mixed solvent of chloroform and ethanol with a volume ratio of 45/55(v/v) and a concentration of 5 wt %; medical polyurethane solution is prepared by a solvent of dimethyl sulfoxide with a concentration of 9 wt %, wherein the voltage is 16 kV, the advance speed is 0.4 ml/h, the acceptable distance is 14 cm, and the inner diameter of pinhole is 0.4 mm. At room temperature of 23° C., in the fume cupboard, the tube portion uses medical polyurethane solution for electrospinning and the ampulla portion uses medical polylactic acid solution for electrospinning

### Embodiment Two

Medical polylactic acid solution is prepared by a mixed solvent of chloroform and ethanol with a volume ratio of 50/50(v/v) and a concentration of 6 wt %; medical polyurethane solution is prepared by a solvent of dimethyl sulfoxide with a concentration of 7 wt %, wherein the voltage is 18 kV, the advance speed is 0.5 ml/h, the acceptable distance is 16 cm, and the inner diameter of pinhole is 0.3 mm. At room temperature of 25° C., in the fume cupboard, the tube portion uses medical polyurethane solution for electrospinning and the ampulla portion uses medical polylactic acid solution for electrospinning.

### Embodiment Three

Medical polylactic acid solution is prepared by a mixed solvent of chloroform and ethanol with a volume ratio of 45/55(v/v) and a concentration of 5 wt %; medical polyurethane solution is prepared by a solvent of dimethyl sulfoxide with a concentration of 9 wt %, wherein the voltage is 19 kV, the advance speed is 0.4 ml/h, the acceptable distance is 14 cm, and the inner diameter of pinhole is 0.3 mm. Medical polyurethane solution is prepared by a solvent of N,N-dimethyl acetamide with a concentration of 20 wt %, wherein the voltage is 19 kV, the advance speed is 2.8 ml/h, the acceptable distance is 20 cm, and the inner diameter of pinhole is 0.3 mm. At room temperature of 24° C., in the fume cupboard, the inner layer uses medical polylactic acid solution for electrospinning and the outer layer uses medical polyurethane solution for electrospinning.

### Embodiment Four

Medical polylactic acid solution is prepared by a mixed solvent of chloroform and ethanol with a volume ratio of 45/55(v/v) and a concentration of 5 wt %; medical polyurethane solution is prepared by a solvent of dimethyl sulfoxide with a concentration of 9 wt %, wherein the voltage is 17 kV, the advance speed is 0.4 ml/h, the acceptable distance is 14 cm, and the inner diameter of pinhole is 0.3 mm. Medical polyurethane solution is prepared by a solvent of THF and dimethyl formamide with a volume ratio of 50/50(v/v) and a concentration of 10 wt %, wherein the voltage is 25 kV, the advance speed is 2.8 ml/h, the acceptable distance is 25 cm, and the inner diameter of pinhole is 0.3 mm. At room temperature of 23° C., in the fume cupboard, the inner layer uses medical polylactic acid solution for electrospinning and the outer layer uses medical polyurethane solution for electrospinning.

### Embodiment Five

Medical ε-poly caprolactone is prepared by a solvent of chloroform with a concentration of 12.5 wt %, wherein the voltage is 15 kV, the acceptable distance is 20 cm, the advance speed is 0.6 ml/h, the spinning time is 3 h, and the spindle speed is 60 Hz. Medical polylactic acid solution is prepared by a solvent of chloroform and dimethylformamide with a volume ratio of 80/20(v/v) and a concentration of 14 wt %, wherein the voltage is 15 kV, the acceptable distance is 20 cm, the advance speed is 1.8 ml/h, the spinning time is 1 h, and the spindle speed is 180 Hz. At room temperature of 25° C., in the fume cupboard, multilayer electrospinning is conducted, the outer is medical ε-poly caprolactone, and the inner layer is medical polylactic acid.

### Embodiment Six

The inner tube solution is dextran with a concentration of 52 wt %, and the outer tube solution is polyethylene glycol-b-poly (L-lactide-co-ε-caprolactone) with a concentration of 19 wt %, the mixed solvent is chloroform ethanol, trifluoroethanol, and N,N-dimethyl formamide with a volume ratio of 58/36/6(v/v/v), wherein the voltage is 13 kV, a flow rate of solution in the inner tube is 0.16 ml/h, the flow rate of solution in the outer tube is 0.6 ml/h, the acceptable distance is 15cm, and at room temperature of 25° C., in the fume cupboard, coaxial electrospinning is conducted.

### Embodiment Seven

Medical ε-poly caprolactone is prepared by a solvent of chloroform with a concentration of 12.5 wt %, which is further concentrated to 65 wt %. Medical polylactic acid is prepared by a solvent of chloroform and dimethylformamide with a volume ratio of 80/20(v/v) and a concentration of 14 wt %, which is further concentrated to 75 wt %. The inner and outer diameters of the inner tube of the spinning nozzle are respectively 0.6 mm and 0.8 mm. The inner and outer diameters of the outer tube are respectively 1.0 mm and 2.0 mm. During spinning, the medical ε-poly caprolactone and medical polylactic acid are respectively injected into the inner tube and outer tube to form coaxial spinning liquid flow. The advance speed of spinning solution in the skin core layer is 0.24 ml/h, the winding speed is 1 cm/s, the acceptable distance is 10 cm, and at room temperature of 25° C., in the fume cupboard, coaxially dry electrospinning is conducted.

### Embodiment Eight

0.5 g of PEG400 and 5 ml diluted saline are for standby use. 20 healthy male SD rats, weighted 231±16.72 g, are divided into 2 groups randomly, namely a group with internal covering membrane of duodenum implanted and the other group with no implantation. Prepare pentobarbital sodium 50 mg/kg ip for the group implanted with the internal covering membrane, implant the internal covering membrane into the duodenum by a conveyor, cut to open the abdomens of rats in both 2 groups, ligate the starting end of the jejunum, raise the breast parts of rats, and slowly inject PEG400 diluent into the bottom of the pylorus by a puncture needle until mild filling. Pay attention to intraoperative care. After 30 min, draw blood from the carotid artery, measure PEG400 by high performance liquid chromatography, analyze the data obtained according to statistics by adopting SPSS 12.0 statistical package. Variance analysis is adopted between groups, and t-test is adopted within groups. Difference makes sense when p is less than 0.05. It proves that the content in the group implanted with internal covering membrane is 0.42±0.13 μg/ml, and the content in the group with no implantation is 5.15±0.66m/ml, p<0.01.

### Embodiment Nine

20 healthy male SD rats, weighted 209±12.43 g, are divided into 2 groups randomly, namely a group with internal covering membrane of duodenum implanted and the other group with no implantation. 10 days after implantation, kill 2 groups of the rats, take out the duodenums and put them into 3.7% paraformaldehyde to fix; wash the fixed tissue blocks by running water thoroughly; dehydrate by the ascending gradient alcohol, make transparent by xylene, wax by paraffin, embed flat with paraffin, remove redundant wax on both sides; cut into slices, flat the slices in warm water, choose the complete and creaseless slices and paste them on the slides; dry out redundant water and paraffin on the slices in a incubator of 55° C., dewax, use downward gradient alcohol; dye with routine HE, dehydrate by the ascending gradient alcohol, make transparent by xylene, conduct mounting by neutral balsam; take 5 discontinuous slices of each rat, take odd-numbered view of each slice in order, and observe small intestinal mucosa and its 20 villi. According to the results of examination with an ordinary optics microscope, villi under optics microscope are basically normal in the examined 2 groups, with a few having broadened intermittence under the top of partial villi. No obvious epithelial peeling, falling off or rupture on the top of villi has been found. No damage or epithelium falling-off on the top of villi, no inherent membrane collapse, and no anabrosis or bleeding point have been found. The data obtained according to statistics are analyzed by adopting SPSS 12.0 statistical package. Variance analysis is adopted between groups, and t-test is adopted within groups. Difference makes sense when p is less than 0.05. The difference between 2 groups under light microscope has no significance (p>0.05).

### Embodiment Ten

12 healthy male SD rats, weighted 221±15.79 g, are divided to 2 groups randomly, namely a group with internal covering membrane of duodenum implanted and the other group with no implantation. 20 days after implantation, kill 2 groups of the rats, take out the duodenums and put them into 3.7% paraformaldehyde to fix; wash the fixed tissue blocks by running water thoroughly; dehydrate by the ascending gradient alcohol, make transparent by xylene, wax by paraffin, embed flat with paraffin, remove redundant wax on both sides; cut into slices, flat the slices in warm water, choose the complete and creaseless slices and paste them on the slides; dry out redundant water and paraffin on the slices in a incubator of 55° C., dewax, use downward gradient alcohol; dye with routine HE, dehydrate by the ascending gradient alcohol, make transparent by xylene, conduct mounting by neutral balsam; take 5 discontinuous slices of each rat, take odd-numbered view of each slice in order, and observe small intestinal mucosa and its 20 villi. Use a transmission electron microscope of 8000 times to take 10 images of each one randomly. No obvious chorionic shortening, lodging or reduced villi absorption area is examined under the transmission microscope, mitochondria in intestinal mucosa cells are basically intact, and no obvious mitochondria swelling, no cell nucleus chromatin condensing and nuclear fragmentation, and no obvious apoptosis are examined. The image processing software is the special software DigitalMicrograph of GATAN company. The data obtained according to statistics are analyzed by adopting SPSS 12.0 statistical package. Variance analysis is adopted between groups, and t-test is adopted within groups. Difference makes sense when p is less than 0.05. The difference between 2 groups has no significance (p>0.05).

The parts not involved in the present invention contain the same prior art, or they may be realized by the prior art.

1. An internal covering membrane of duodenum, wherein the membrane is prepared from biocompatible materials via electrospinning technology, being able to prevent food from contacting with the intestinal mucosa in anatomy without affecting the physiological functions of intestinal mucosa cells.

2. The internal covering membrane of duodenum according to claim 1, wherein the membrane comprises an ampulla portion and a tube portion, and its thickness is 1 μm-1 mm, wherein an outer surface circumference of the ampulla portion is provided with elastic fibers with anchoring means.

3. The internal covering membrane of duodenum according to claim 1, wherein the membrane is produced by the following:
(1) devices and instruments being: a high-voltage electrostatic generator, a micro-injection pump, 1-10 spinning nozzles, a roller or a plate receiving device, a fiber fineness meter, a rotary viscometer, a surface tension meter, a conductivity meter, a digital vacuum scanning electron microscope and a fume cupboard;
(2) solution preparation being: preparing medical polylactic acid solution with a concentration of 1 wt %-35 wt %, wherein a solvent is a mixture of chloroform and ethanol, with a volume ratio of 25/75(v/v)-100/0(v/v); preparing medical polyurethane solution with a concentration of 1 wt %-45 wt %, wherein the solvent is dimethyl sulfoxide;
(3) technological parameter being: spinning voltage: 10-36 kV, advance speed: 0.1-3.5 ml/h, acceptable distance: 10-28 cm, inner diameter of pinhole: 0.1-1 mm;
(4) electrospinning process being: flattening a needle head, in the fume cupboard fixing a syringe injected with electrospinning solution on the micro-injection pump, connecting the output cable of the high-voltage electrostatic generator to the metal needle on the front end of the syringe, making the receiving device grounded, adjusting the distance between the receiving device and a capillary, starting the micro-injection pump, adjusting a flow rate of the injection pump, gradually increasing the voltage after a stable hemispherical droplet, that is Taylor cone, is formed in the capillary orifice, and collecting electrospinning fibers in the form of a non-woven membrane on the receiving device; wherein
the tube portion is able to use medical polyurethane solution for electrospinning and the ampulla portion is able to use medical polylactic acid solution for electrospinning.

4. The internal covering membrane of duodenum according to claim 1, wherein the electrospinning solution is prepared by the following high polymer materials: polylactic acid, polyurethane, PEUR Poly(ether urethane), polyether sulfone, polyglutamic acid, polyvinyl alcohol, polyhydroxybutyrate, caprolactone, polycaprolactone, polyhydroxybutyrate, polyvinylpyrrolidone, poly-L-lactide, recombinant spider silk protein, amino acids, polycaprolactone, caprolactam, hydroxyapatite, elastin, heparin, and glycolic acid.

5. The internal covering membrane of duodenum according to claim 1, wherein the membrane can be formed by blending electrospinning or/and multilayer electrospinning or/and core-shell electrospinning or/and dry electrospinning.

6. The internal covering membrane of duodenum according to claim 1, wherein the membrane is used to manufacture medical devices for treating diabetes and obesity, with functions of reducing damage, preventing falling-off, avoiding removal and inhibiting bounce.
